SEQUENCE LISTING (12 sequences)

SEQ ID NO: 1 - DNA, 15 bp, Artificial
  Sense oligonucleotide to human retinol binding protein.
gtcactcccgaaatg

SEQ ID NO: 2 - DNA, 15 bp, Artificial
  Antisense oligonucleotide to human retinol binding protein.
catttcgggagtgac

SEQ ID NO: 3 - DNA, 24 bp, Artificial
  Sense oligonucleotide to human folic acid binding protein.
gggacagacatggctcagcggatg

SEQ ID NO: 4 - DNA, 24 bp, Artificial
  Antisense oligonucleotide to human folic acid binding protein.
catccgctgtgccatgtctgtccc

SEQ ID NO: 5 - DNA, 24 bp, Artificial
  Sense oligonucleotide to human riboflavin carrier protein.
ggaacaacaatgctgaggtttgcc

SEQ ID NO: 6 - DNA, 24 bp, Artificial
  Antisense oligonucleotide to human riboflavin carrier protein.
ggcaaacctcagcattgttgttcc

SEQ ID NO: 7 - protein, 182 aa, Homo sapiens
  misc_feature: Retinol binding protein.
ERDCRVSSFRVKENFDKARFSGTWYAMAKKDPEGLFLQDNIVAEFSVDETGQMSATAKGRVRLLNNWDVCADMVGTFTDTEDPAKFKMKYWGVASFLQKGNDDHWIVDTDYDTYAVQYSCRLLNLDGTCADSYSFVFSRDPNGLPPEAQKIVRQRQEELCLARQYRLIVHNGYCDGRSERNL

SEQ ID NO: 8 - protein, 292 aa, Rattus norvegicus
  misc_feature: Folic acid binding protein.
VDSVYRTRSLGVAAEGIPDQYADGEAARVWQLYIGDTRSRTAEYKAWLLGLLRQHGCHRVLDVACGTGVDSIMLVEEGFSVTSVDASDKMLKYALKERWNRRKEPAFDKWVIEEANWLTLDKDVPAGDGFDAVICLGNSFAHLPDSKGDQSEHRLALKNIASMVRPGGLLVIDHRNYDYILSTGCAPPGKNIYYKSDLTKDITTSVLTVNNKAHMVTLDYTVQVPGAGRDGAPGFSKFRLSYYPHCLASFTELVQEAFGGRCQHSVLGDFKPYRPGQAYVPCYFIHVLKKTG

SEQ ID NO: 9 - protein, 222 aa, Bos taurus
  misc_feature (55)..(55): Xaa at location 55 is unknown.  Milk folate binding protein.
AQAPRTPRARTDLLNVCMDAKHHKAEPGPEDSLHEQCSPWRKNACCSVNTSIEAXKDISYLYRFNWDHCGKMEPACKRHFIQDTCLYECSPNLGPWIREVNQRWRKERVLGVPLCKEDCQSWWEDCRTSYTCKSNWHKGWNWTSGYNQCPVKAAHCRFDFYFPTPAALCNEIWSHSYKVSNYSRGSGRCIQMWFDPFQGNPNEEVARFYAENPTSGSTPQGI

SEQ ID NO: 10 - protein, 257 aa, Homo sapiens
  misc_feature: Folate binding protein precursor.
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 11 - protein, 222 aa, Bos taurus; identical to SEQ ID NO: 9
  misc_feature (55)..(55): Xaa at location 55 is unknown.  Folate binding protein.

SEQ ID NO: 12 - protein, 245 aa, Homo sapiens [sequence omitted]

(19) United States
(12) Patent Application Publication
(10) Pub. No.: US 2004/0214788 A1
(43) Pub. Date: Oct. 28, 2004

(54) Treatment of prostate cancer

(76) Inventors: Madhwa H.G. Raj, Metairie, LA (US); Prakash N. Rao, Kenner, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/797,690

(22) Filed: Mar. 10, 2004

Related U.S. Application Data
(60) Provisional application No. 60/453,602, filed on Mar. 11, 2003.

(51) Int. Cl. A61K 48/00

(52) U.S. Cl. 514/44

(57) ABSTRACT

A method is disclosed for inhibiting the growth of prostate cancer by administering to a patient an antagonist against one or more of the vitamin binding proteins riboflavin carrier protein, retinol binding protein, and folic acid binding protein. The antagonist may, for example, be an antibody against the vitamin binding protein, an antibody against the corresponding vitamin binding protein receptor, or a conjugate of the corresponding vitamin and an immunogen that stimulates immune response. In the latter case the immunogen may, for example, be an immunogen that has previously been used to immunize the patient. Heterologous vitamin binding proteins may be used to actively immunize patients. The antagonist may also be an antisense oligonucleotide or a double-stranded RNA molecule that inhibits cellular expression of vitamin binding proteins.

The benefit of the Mar. 11, 2003 filing date of provisional application No. 60/453,602 is claimed under 35 U.S.C. § 119(e).

This invention pertains to methods for treating prostate cancer.

Prostate cancer is the most common cancer leading to death among United States men. Its incidence has increased considerably over the past two decades. This cancer is often initially responsive to hormone treatment, but then frequently progresses into a hormone-insensitive state that is not easily treated. Further, once the cancer spreads or metastasizes from the prostate gland to other tissues such as bone marrow, the cancer usually cannot be cured by presently available treatments, which are either too toxic or cannot reach the metastases. There is a dire need for new strategies to treat prostate cancers, including metastasized prostate cancers, with low toxicity.

A vitamin binding protein (VBP) is a protein with a high-affinity for its target vitamin. VBPs are present throughout the animal kingdom and play a vital role in various life processes. Their functions include the storage and transport of vitamins, and the prevention of rapid losses of these vital micronutrients due to excretion or metabolic degradation. Several specific vitamin binding proteins have been identified, including riboflavin carrier protein (RCP); thiamin carrier protein (TCP); biotin, a binding protein for vitamin B12 (transcobalamin); retinol binding protein (RBP), a binding protein for vitamin D; and folic acid binding protein (FBP). The carriers for vitamin D, retinol, and folic acid are expressed constitutively. Expression of the carriers for riboflavin, thiamin, and biotin is induced de novo as a reproductive strategy in birds, to facilitate vitamin deposition in the developing egg. In the rat, levels of RCP increase during pregnancy. It has been shown that immunoneutralization of this carrier protein by specific antibodies terminates pregnancy in the rat.

RCP, RBP, FBP, TCP, and transcobalamin have each been characterized. Each is a high molecular weight protein. The primary structures of RCP, RBP, and FBP and their DNA coding sequences have also been reported.

Some vitamin binding proteins have been reported to play a role in certain cancers. FBP has been reported to be overexpressed in ovarian, endometrial, breast, lung, colorectal, and renal cell cancers. See M. Wu et al., “Expression of folate receptor type alpha in relation to cell type, malignancy and differentiation in ovary, uterus and cervix,” Cancer Epidemiol. Biomarkers. Prev., vol. 8, pp. 775-782 (1999); M. Pavlovic et al., “Altered transport of folic acid and antifolates through the carrier mediated reduced folate transport system in a human leukemia cell line resistant to (6R)5,10-dideazatetrahydrofolic acid (DDATHF),” Adv. Exp. Med. Biol., vol. 338, pp. 775-778(1993); and I. Campbell et al., “Folate binding protein is a marker for ovarian cancer,” Cancer Res., vol. 51, pp. 5329-5338 (1991).

M. Raj et al., “Evaluation of riboflavin carrier protein (RCP) as a serum and tumor marker of ovarian epithelial cancer,” Proc. Amer. Assoc. Cancer. Res., vol. 42, p. 46, abstr #242 (2001) disclosed that RCP may be used as a marker for detecting breast adenocarcinoma, and reported preliminary results showing that RCP appeared to be useful as a marker for ovarian epithelial cancer.

FBP has been used in developing therapeutic strategies for cancers. Bi-specific antibodies both to FBP and to T cell receptors have been shown to stimulate CTLs (cytotoxic T lymphocytes) that target tumor cells See M. Pavlovic et al. (1993); and D. Kranz et al., “Conjugates of folate and anti T cell receptor antibodies specifically target folate receptor positive tumor cells for lysis,” Proc. Natl. Acad. Sci. (USA), vol. 92 pp. 9057-9061 (1995).

R. Corrocher et al., “Specific and non-specific folate binding protein in normal and malignant human tissues,” J. Clin. Pathology, vol. 31, pp. 659-665 (1978) reported that certain leukemia cells bound folic acid more strongly than do normal cells, as did cells from stomach and colon carcinomas to a lesser extent.

It has been suggested that peptide sequences from FBP that are capable of CTL induction might be used as vaccines against epithelial cancers. See G. Peoples et al., “Vaccine implications of folate binding protein, a novel cytotoxic T-lymphocyte recognized antigen system in epithelial cancers,” Clin. Canc. Res., vol. 5, pp.4214-4223 (1999); and D. Kim et al., “Folate binding protein peptide 191-199 presented on dendritic cells can stimulate CTL from ovarian and breast cancer patients,” Anticancer Res., vol. 19, pp. 2907-2916 (1999).

Anti-FBP antibodies have been combined with radionuclides in targeted radioimmunotherapy of ovarian cancer in the nude mouse model. See. H. Anderson et al., “Radioimmunotherapy of nude mice with intraperitoneally growing ovarian cancer xenograft using ²¹¹At labeled monoclonal antibody Mov 18,” Anticancer Res., vol. 20, pp. 459-462 (2000).

Other strategies include targeting interleukin-2 with an anti-folate-receptor antibody, a DNA vaccination strategy for FBP, and inducing CTL and antibody responses in mice with ovarian cancer. See, respectively, C. Melani et al., “Targeting of Interleukin-2 to human ovarian carcinoma by fusion with a single-chain Fv of antifolate receptor antibody,” Cancer Res., vol. 58, pp. 4146-4154 (1998); and F. Neglia et al., “DNA vaccination against the ovarian carcinoma associated antigen folate receptor alpha induces cytotoxic T-lymphocyte and antibody responses in mice,” Cancer Gene Therapy, vol. 6, pp. 349-357 (1999).

The role of RBP in various cancers has been investigated to a lesser extent. Its presence has been demonstrated in brain tumors and retinoblastoma cells. See M. Al-Ubaidi et al., “Bilateral retinal brain tumors in transgenic mice expressing simian virus 40 large T antigen under control of human interphotoreceptor retinoid binding promoter,” J. Cell Biol., vol. 119, pp. 1681-1687 (1992); M. Rodgrigus et al., “Retinoblastoma mRNA for interphotoreceptor retinoid binding protein,” Curr. Eye. Res., vol. 11, pp. 425-433 (1992); and H. Korf et al., “Immunocytochemical demonstration of interphotoreceptor retinoid binding protein in cerebellar medulloblastoma,” Acta Neopathol. (Berl.), vol. 83, pp. 482-487 (1992).

F. Cope et al., “Retinoid receptor antisense DNAs inhibit alkaline phosphatase induction and clonogenicity in malignant keratinocytes,” Proc. Natl. Acad. Sci. USA, vol. 86, pp. 5590-5594 (1988) reported that treatment with antisense oligodeoxynucleotides corresponding either to human cellular RBP or to human nuclear retinoic acid receptor (hnRAR) significantly attenuated the level of cytoplasmic cRBP and hnRAR in a concentration- and time-dependent manner, and blocked the induction of alkaline phosphatase by retinol in human malignant keratinocytes. Treatment with these antisense nucleotides for more than 3 hours induced cell rounding, loss of cell-to-cell attachment, loss of cell adhesion to the substratum, and loss of clonogenic potential for the cells.

In the mouse, the injection into the yolk sac of antisense oligodeoxynucleotides for RBP during embryogenesis has been reported to inhibit the formation of vitelline blood vessels, and to down-regulate the developmentally important genes TGF-β and Shh (Sonic hedgehog), which act as inducing signals in several regions of the embryo. See C. Bavik et al., “Developmental abnormalities in cultured mouse embryos deprived of retinoic acid by inhibition of yolk sac retinol binding protein synthesis,” Proc. Natl. Acad. Sci. (USA), vol. 93, pp. 3110-3114 (1995).

H. Senoo et al., “Transfer of RBP from Hep G2 human hepatoma cells to co-cultured rat stellate cells,” Proc. Soc. Natl. Acad. Sci. USA, vol. 15, pp. 3616-3620 (1993) reported that human hepatoma cells synthesize and secrete RBP, and that there appeared to be a cell surface receptor for RBP. It was reported that RBP was transferred to stellate cells in co-cultures, and that this transfer could be blocked by the addition of RBP antibodies.

D. Kim et al., “Folate binding protein peptide 191-199 presented on dendritic cells can stimulate CTL from ovarian and breast cancer patients,” Anticancer Research, vol. 19, pp. 2907-2916 (1999) reported that folate binding protein is overexpressed in over 90% of ovarian and 20-50% of breast cancers, and that folate binding protein was the source of antigenic peptides recognized by tumor-associated lymphocytes. It was also reported that stimulation with a 9-amino acid peptide caused an enhanced CTL response. This CTL reaction has also been reported to result from inoculating with DNA that encodes FBP.

F. Neglia et al., “DNA vaccination against the ovarian carcinoma-associated antigen folate receptor α (FRα) induces cytotoxic T lymphocyte and antibody responses in mice,” Cancer Gene Therapy, vol. 6, pp. 349-357 (1999) discloses that human folate receptor α is a folate-binding protein that is overexpressed in ovarian carcinoma. DNA immunization with plasmid DNA encoding FRα was reported to induce the production of antibodies in mice, and to significantly reduce the in vivo growth of syngeneic C26 colon cancer cells inoculated into the mice.

G. Peoples, “Vaccine implications of folate binding protein, a novel cytotoxic T lymphocyte-recognized antigen system in epithelial cancers,” Clinical Cancer Research, vol. 5, pp. 4214-4223 (1999) disclosed that FBP is a source of antigenic peptides recognized in both breast cancer and ovarian cancer. It was reported that certain peptide epitopes of FBP were efficient at amplifying the response of tumor-associated lymphocyte populations. This paper said that the distribution of FBP among a large fraction of ovarian, endometrial, breast, lung, colorectal, and renal carcinomas suggested the possibility of using FBP-based vaccines against these cancers.

We have previously reported that RCP is over-expressed in breast adenocarcinoma, and that elevated serum levels can be used as a marker for diagnosis of early (stage 1) breast cancer, as well as liver, ovarian, and endometrial cancers. See P. Rao et al., “Elevation of serum riboflavin carrier protein in breast cancer,” Cancer Epidemiology Biomarkers. Prev., vol. 8, pp. 985-990 (1999). See also our earlier U.S. Pat. No. 6,197,532.

U.S. Pat. No. 5,547,668 discloses a process of targeting folate-receptor positive tumor cells, such as those from ovarian tumors, for lysis by binding a conjugate of folate and an anti-T-cell-receptor antibody on an anti-Fc receptor antibody to those cells.

Published U.S. patent application Ser. No. 2001/0031252 discloses a method for enhancing the endogenous immune response of an animal to pathogenic cells that preferentially express a binding site for a ligand, by administering to the animal the ligand conjugated to an immunogen that is recognized by the animal's immune system. An additional therapeutic factor may be co-administered, such as an immune system stimulant, for example certain interleukins. For example, inbred mice were immunized with fluorescein isothiocyanate. The mice were then injected with a syngeneic lung cancer cell line that expressed high levels of folate receptor. It was observed that the mice survived longer, in a dose-dependent manner, when they were injected with both fluorescein isothiocyanate and IL-2.

Published international patent application WO 02/15920 discloses the administration of a retinol binding protein antagonist to treat a patient suffering from a hyperproliferative disorder or photoaging. For example, in a prophetic example severe combined immunodeficiency mice are implanted with human renal cell carcinoma samples. After the implanted tumors have grown to a palpable size, the mice are injected with anti-retinol binding protein receptor monoclonal antibody. It was predicted that tumor size would remain the same or decrease, while that in control mice would increase.

G. Prins et al., “Retinoic acid receptors and retinoids are up-regulated in the developing and adult rat prostate by neonatal estrogen exposure,” Endocrinology, vol. 143, pp. 3628-3640 (2002) reported that retinoic acid receptors.(which are distinct from “retinol binding protein”) are localized in the normal rat prostate gland.

A. Colige et al., “Use of antisense oligonucleotide to inhibit expression of a mutated human procollagen gene (COL1A1) in transfected mouse 3T3 cells,” Biochem., vol. 32, pp. 7-11 (1993) discloses that antisense oligonucleotides could be used to inhibit specifically the expression of a mutated exogenous gene for collagen, without inhibiting expression of an endogenous gene for the same protein.

N. Dean et al., “Inhibition of protein kinase C-α expression in mice after systemic administration of phosphorothioate antisense oligodeoxynucleotides,” Proc. Natl. Acad. Sci. USA, vol. 91, pp. 11762-11766(1994) discloses that a 20-mer phosphorothioate antisense oligonucleotide inhibited the expression of protein kinase C-α both in vitro and in vivo.

W. Brysch et al., “Design and application of antisense oligonucleotides in cell culture, in vivo, and as therapeutic agents,” Cell. Molec. Neurobiol., vol. 14, pp. 557-568 (1994) provides a review of the use of antisense oligonucleotides.

To the inventors' knowledge, no information has previously been published on the presence, role, or distribution of any vitamin binding proteins in prostate cancer.

We have discovered a method for inhibiting the growth of prostate cancer, by administering to a patient an antagonist against one or more of the vitamin binding proteins riboflavin carrier protein, retinol binding protein, and folic acid binding protein. The antagonist may, for example, be an antibody against the vitamin binding protein, an antibody against the corresponding vitamin binding protein receptor, or a conjugate of the corresponding vitamin and an immunogen that stimulates immune response. In the latter case the immunogen may, for example, be an immunogen that has previously been used to immunize the patient.

The novel strategy involves attacking prostate cancer by modifying the vitamin binding proteins, for example by using specific antibodies. The vitamins retinol, folic acid, and riboflavin all: (1) are involved in multiple biochemical pathways, including DNA methylation, cell division, cell cycle regulation, and proper endothelial function; (2) are needed by rapidly-dividing prostate cancer cells; (3) cannot be synthesized by the cancer cells, and must instead be obtained from outside sources; and (4) have binding proteins that are up-regulated and overexpressed by prostate cancer cells, possibly as a mechanism for the cancer cells to obtain the needed high levels of the vitamins.

In a preferred embodiment, the therapy deprives prostate cancer cells of one or more of three essential vitamins: vitamin A, also called retinol, and two vitamins that belong to the vitamin B group, namely folic acid and riboflavin. Vitamin A is essential for cellular growth. Folic acid is essential for cell division and DNA synthesis. Riboflavin is important for the proper functioning of blood vessels, and in certain reactions that provide energy to cells. The need for these vitamins is especially critical in rapidly growing tissues such as tumors.

We are studying in detail the abnormal expression patterns of the three vitamin binding proteins folate binding protein (FBP), retinol binding protein (RBP), and riboflavin carrier protein (RCP) in both androgen-sensitive and androgen-insensitive prostate cancer cell lines, as well as in tumor samples surgically removed from patients. We have used androgen-sensitive and androgen-insensitive prostate cancer cell lines in tissue culture to investigate the effects of depriving the cells of these vitamins, for example by using antibodies or antisense oligonucleotides. Optionally, antisense oligonucleotides may comprise modified nucleotides that render the oligonucleotides more resistant to in vivo degradation by RNase than are otherwise identical oligonucleotides consisting only of unmodified nucleotides. For example, we have used specific antibodies to the vitamin binding proteins, both individually and in combination, to measure their effectiveness in preventing prostate cancer tumor formation and to cause tumor regression in the nude mouse model. Preliminary results have indicated that both of these goals can be achieved without significant toxic side effects. We have made antibodies to these binding proteins and shown that the antibodies kill prostate cancer cells in vitro. We have also tested these antibodies in the nude mouse model. Nude mice were injected with human prostate cancer cells, and thereafter developed tumors. However, administering antibodies to the vitamin binding proteins prevented the formation of prostate cancer tumors in the mouse. Further experiments demonstrate the ability of these antibodies to decrease tumor size, or to completely cure the cancer.

This invention enables the production of potent vaccines against, and the administration of powerful immunotherapies against, both hormone-sensitive and hormone-resistant varieties of prostate cancer, as well as the treatment of metastasized disease, all with low toxic side effects. The vaccines may work by antibody production, cellular immune mechanisms (e.g., cytotoxic T lymphocyte production), or both.

Preliminary results. We have, for the first time, immunohistochemically localized FBP in prostate cancer tissue, and have observed that it is highly over-expressed as compared to FBP levels in normal prostate tissue.

We have previously observed over-expression of RBP in ovarian and breast adenocarcinomas (Raj and Rao, unpublished results). We have, for the first time demonstrated that FBP and RBP are over-expressed in prostate adenocarcinomas as compared to expression in normal prostate glands.

We have also observed, for the first time, moderate over-expression of RCP in prostate adenocarcinomas as compared to levels in normal controls.

In these preliminary studies, we used specific antibodies to human urinary RBP, bovine milk FBP, and chicken egg white RCP. Each of these antibodies was produced in rabbits by active immunization with the respective antigen. The resulting antibodies were absorbed against solid-phase serum proteins, in order to remove non-specific antibodies to serum proteins, according to the method of S. Avrameas et al., “Biologically active water-insoluble protein polymers,” J. Biol. Chem., vol. 7, pp. 1851 ff (1967). The gamma globulin fraction was isolated by ammonium sulfate fractionation, and was then purified on a protein A-Sepharose column. Using a radioimmunoassay, we have demonstrated that the anti-RCP antibodies did not cross-react with FBP or RBP.

These experimental results also demonstrated the immunogenicity of heterologous vitamin binding proteins. In other words, a vitamin binding protein from one species (or a peptide subunit of such a vitamin binding protein) may be used to immunize a different species, which will then produce antibodies. Those antibodies will not only bind to the heterologous vitamin binding protein, but will also cross-react with the homologous “self” vitamin binding protein. This immunogenicity has implications for making vaccines against the cancers, as discussed further below.

We have demonstrated the effectiveness of antibodies to RCP, RBP, and FBP against prostate cancer cells in tissue culture. LNCAP prostate cancer cells were grown in culture dishes to about 50% confluence, and anti-RBP IgG or anti-FBP IgG was then added at a concentration of 0.2 mL/mL of medium. Non-immune IgG from rabbit serum was added to control dishes. Addition of specific anti-RCP antibodies, anti-FBP antibodies, or anti-RBP antibodies induced 100% cell detachment and rounding within 24 hours. The detached cells were nonviable, as evaluated by the trypan blue dye exclusion test. By contrast, cells in control dishes treated with non-specific IgG continued to grow normally, and appeared to be healthy. We have performed cell-killing experiments using antibodies against each of RCP, RBP, and FBP, and found each to be effective. Each of the three antibodies against the vitamin-binding proteins was effective in killing both androgen sensitive (LNCAP) and androgen-insensitive (PC-3) prostate cancer cells in culture.

This demonstration that anti-RCP, anti-RCP, and anti-RBP antibodies can kill cancer cells is believed to be the first of its kind for any type of cancer cells, and in particular is believed to be the first such demonstration that anti-FBP antibodies can kill prostate cancer cells. These preliminary studies are being extended to androgen-resistant cell lines such as DU-145 and PC-3.

In another preliminary study, LNCAP tumors were developed in nude mice by subcutaneously injecting 5×10⁶ cells in 0.1 mL of Matrigel™ extracellular matrix preparation into the flanks. Two to four weeks later tumors appeared in 9 of 10 control animals at the site of injection. Three other mice (treatment group) were given twice-weekly intraperitoneal injections of a mixture of anti-RBP, anti-FBP, and anti-RCP antibodies (0.2 mL each), beginning at the same time as an otherwise identical challenge with the tumor cells. Tumor formation was inhibited in all the three of the mice so treated. These experiments are being repeated in a larger group of mice. Also, we are examining the effect of administering the three individual anti-VBP antibodies separately, and of administering each of the three possible pairwise combinations of the three antibodies.

Without wishing to be bound by this theory, the following is our current hypothesis as to the underlying mechanism of action. We hypothesize that vitamin binding proteins are over-expressed in both hormone-sensitive and hormone-insensitive prostate cancers, and that these VBPs are obligatory for the transport of vitamins that are critically needed by the prostate cancer cells to sustain the fast pace of cell division that typifies tumor growth. Based on our preliminary results we further hypothesize that deprivation of these vitamins leads to cell death once the cell has initiated division. However, resting cells in interphase may not require such high levels of vitamins, and may not be similarly affected. Thus, we hypothesize that the neutralization of VBPs selectively affects rapidly dividing tumor cells, without toxicity to most normal cells.

### EXAMPLES 1-3

Production and characterization of polyclonal antibodies to RBP, FBP and RCP. RBP, FBP, and RCP were purchased from Sigma Chemical Co., St. Louis, Mo. Our results have shown that each of these antigens is immunogenic, and that each stimulated the production of antibodies when injected into a different species.

Adult male New Zealand White rabbits bred specifically for immunization were used in these experiments. All appeared to be free from infectious disease both before and during the experiments. These rabbits were immunized with 1 mg of the particular antigen, dissolved in 0.5 mL of distilled water, emulsified with an equal volume of Freund's complete adjuvant, and injected subcutaneously at multiple sites on the back of each animal. Booster injections of the antigen in Freund's incomplete adjuvant were given at monthly intervals. The animals were test bled from the ear vein, starting three months after the initial immunization. After antibody titers had reached sufficient levels, the animals were bled monthly to collect sufficient antiserum for preparation of the gamma globulin fraction and characterization. In our experience, antibody titers are generally sufficiently high by the third month, as all three of these VBPs are strong antigens. Sufficiency of antibody titer is judged by quantitative precipitin test and ELISA. As a rule of thumb, we usually consider as sufficient an antibody titer of about 1 mg/mL serum or higher.

Batches of antiserum from each rabbit were pooled and absorbed onto a solid phase normal serum prepared by the method of S. Avrameas et al. (1967). The normal serum was derived from the same species as the antigen (viz., chicken serum for RCP, bovine serum for FBP, and human serum for RBP). This procedure resulted in the essentially complete removal of any non-specific antibodies. The gamma globulin fraction was obtained by ammonium sulfate fractionation, and was further purified on a Protein A-Sepharose column using standard procedures. The purified antibody fraction was reconstituted to a 2× to 4× concentration, sterile-filtered with a 0.2 micron filter, and stored in aliquots at −25° C. The resulting antibodies were polyclonal, but monospecific. We have demonstrated by radioimmunoassay that none of RCP, FBP, and RBP cross-react with specific antibodies for either of the other two VBPs (data not shown).

A pooled batch of pre-immune serum from the same rabbits was otherwise processed identically to obtain a non-immune gamma globulin fraction, for use in control experiments.

### EXAMPLES 4-6

Antibody characterization. The antibodies were characterized using agar gel double diffusion and Western blot analysis using standard procedures. The antibody content was quantified using a quantitative precipitin test, expressed as mg of specific antibody/mL serum. Antibody titers are assessed by ELISA, and antibody specificity was characterized using Western blots with the specific antibody used to develop the bands.

### EXAMPLES 7-9

Evaluation of expression patterns of RBP, FBP and RCP in prostate cancers. Immunohistochemical localizations of RBP, RCP and FBP were performed on both normal prostate tissue and prostate cancer tissue that had been collected during surgery and archived in a pathology laboratory. The tissues were fixed in formalin and blocked in paraffin. Tissues thus preserved have been found to be well-suited for analysis of expression patterns, as their antigens are very stable and yield consistent results by immunohistochemical methods. Five micron sections of the tissue were cut, and adjacent sections were chosen for localizing each of the three VBPs. Briefly, the sections were deparaffinized in xylene and were then hydrated. Nonspecific sites were blocked using normal goat serum, and any endogenous peroxidases were inactivated by incubating with 0.3% hydrogen peroxide. The tissue sections were then treated with primary antibody to FBP, RBP, or RCP. The sections were washed and treated with biotinylated goat anti-rabbit gamma globulin, avidin-peroxidase conjugate, and chromogen substrate to develop a red or brown color. The sections were counterstained with hematoxylin, dehydrated, and mounted. Control specimens were also prepared, by omitting the primary antibody, or by substituting non-immune rabbit gamma globulin. These techniques are standardized by means used in the art to yield consistent results between experiments. The sections are examined and photographed with a Nikon Labphot microscope. Digital images are acquired with a Micromax cooled CCD digital camera system (Advanced Imaging Corporation), and are analyzed using MetaMorph software, version 4.5 (Advanced Scientific). Sections are analyzed from each of ten normal samples, and from ten cancer samples taken at each of the following four stages: A (occult cancer), B (cancer nodule confined within the prostatic capsule), C (cancer with extracapsular extension into surrounding structures; or confined within the capsule, but with elevation of serum acid phosphatase, or involvement of pelvic lymph nodes, and D (demonstrated bone or extra-pelvic involvement). We expect the results to show an increased level of expression of each of the VBPs in cancer tissues as compared to controls.

Further, a folate receptor similar to FBP has been reported on cancer cell membranes. However, to the inventors' knowledge, analogous receptors, similar to RBP and RCP, have not previously been reported on cancer cell membranes. Without wishing to be bound by this theory, we hypothesize that such analogous receptors do exist. We will demonstrate their existence by observing, for example, the binding of radiolabelled antibodies to RBP and RCP to prostate cancer cell membranes.

Effects of antibodies to RBP, FBP, and RCP on prostate cancer cells in tissue culture.

### EXAMPLES 10-13

Tissue cultures. The androgen-sensitive LNCAP prostate cancer cell line (American Type Culture Collection, ATCC) was cultured in RPMI-1640 medium containing glutamine and 10% fetal calf serum (FCS) in 5% CO₂-air at 37° C. The two androgen insensitive cell lines DU-145 and PC-3 (ATCC) were grown in DMEM medium with glutamine and 10% FCS.

Cell growth and viability. At the end each experiment, any detached cells are harvested and tested for viability by the trypan blue dye exclusion test. Briefly, 1 mL of a 1% dye solution is mixed with 0.5 mL of medium containing the cells. A sample is loaded onto a hemocytometer and cells are counted under a microscope. The numbers of dead and live cells are recorded. Any attached cells are first trypsinized using standard techniques, and are then subjected to the dye exclusion test. Inhibition of growth is calculated by comparing the numbers of live and dead cells in the control dishes with those in the antibody-treated dishes.

PSA immunoassay. Secretion of prostate-specific antigen (“PSA”) in the culture medium is quantitated as a measure of cell function. A commercially available kit is used for the PSA assay, according to the manufacturer's instructions.

Experimental design and statistical analysis. Prostate cancercells grown in T-75 flasks are trypsinized, counted, seeded at the rate of 1 million cells/dish, and are then grown to about 50% confluence. Appropriate antibody gamma globulin (purified) to either RBP, FBP, or RCP is sterilized with a 0.22 micron filter, and is then added to the cells to a total volume of 3 mL. Each group is repeated in triplicate. Three different types of controls are used to ensure that the observed effects are indeed attributable to the specific antibodies: (1) Controls treated with non-immune gamma globulin, (2) controls treated with heat-inactivated immune gamma globulin (heat-treated in a boiling water bath for 30 minutes), and (3) controls treated with an antigen-absorbed antibody fraction.

Based on our preliminary observations that cell detachment and killing occurred by 24 hours after treatment, time-course measurements are made after 2, 4, 8, 16, and 24 hours of incubation. At each measurement, the cells are assessed for viability and growth, and the medium is assayed for PSA. Statistical comparisons are performed using ANOVA (analysis of variance) and Student's t-test. Statistical significance is set at P<0.05.

These experiments and measurements are repeated for LNCAP, DU-145, PC-3, and normal (control) prostate cell lines, to demonstrate the effectiveness of neutralizing the VBPs as a treatment for both androgen-sensitive and androgen-resistant tumor cell lines.

Expected results. Based on our preliminary findings, we expect the results to show that cell growth and viability are affected by one or more of the anti-RCP, anti-RBP, and anti-FBP antibodies. We further expect to observe these effects in a progressively time-dependent manner. We also expect to see the effects of the antibodies manifested in both androgen-sensitive and androgen-insensitive cell lines.

### EXAMPLES 14-17

Confirmation of proposed mechanism of action. In view of the rapid onset of changes following antibody treatment, we examine whether the antibody-treated cells undergo apoptosis. For this purpose, we perform one set of time course experiments with each cell line. Harvested cells are assessed for apoptosis using the cell death detection ELISA plus kit (Boehringer Mannheim, Indianapolis, Ind.), which quantitates histone-associated DNA fragments in the cytoplasm of the cells. Apoptosis is also confirmed visually with the TUNEL method (TdT-mediated dUTP nick end labeling) (In Situ Cell Death Detection Kit, Boehringer Mannheim). This method identifies DNA strand breaks in the early steps of apoptosis. See Y. Gavrieli et al., “Identification of programmed cell death in situ via specific labeling of nuclear DNA fragmentation,” J. Cell. Biol., vol. 119, pp. 493-501 (1992).

To demonstrate that the antibodies inhibit the uptake of the ligand vitamin, we perform an additional set of time course experiments with each cell line, generally as described above. At the beginning of the experiment 1 μCi of the appropriate ³H-labeled vitamin (i.e., retinol, folic acid or riboflavin) is added per dish. The cells are harvested at the end of the experiment and washed, and labeled vitamin uptake is determined by counting in a Packard β spectrometer. Each group is repeated in triplicate. For comparison, controls are treated with non-immune globulin, or denatured antibody, or absorbed antibody. We expect to find that tritiated vitamin uptake is inhibited by the antibody to the corresponding binding protein.

### EXAMPLES 18-23

Effects of inhibiting cellular expression of VBPs in prostate cancercells in vitro. Preliminary results have shown that cellular expression of the VBPs is up-regulated, resulting in intense immunohistochemical staining of malignant cells in prostate tumor samples from patients. The intracellular role of these overexpressed VBPs is not known. While storage of acquired vitamin in a stable form bound to its binding protein is a possibility, it is also possible that the binding proteins may act as transcription factors, or that they may aid in transporting the vitamin to the nucleus, or to other parts of the cell where the vitamin is needed as a co-factor. In order to test these possibilities, we modify the cellular expression of each of the three vitamin binding proteins using antisense oligonucleotides.

### EXAMPLES 18-19

Antisense oligonucleotides to RBP. We synthesize the antisense oligodeoxynucleotide to RBP described by Cope et al. The antisense oligonucleotide is used in a time- and concentration-dependent experimental design to assess its effects on both an androgen-sensitive prostate cancer cell line (LNCAP) and two androgen-insensitive cell lines (DU-145 and PC-3). The sense and antisense oligodeoxynucleotides have the following sequences:

| | | |
|---|---|---|
| sense cRBP: | 5′-GTCACTCCCGAAATG-3′ | (SEQ ID NO:1) |
| antisense cRBP: | 5′-CATTTCGGGAGTGAC-3′ | (SEQ ID NO:2) |

### EXAMPLES 20-21

Antisense oligonucleotides to FBP. To the inventors' knowledge, there are no reports in the literature of using an antisense oligonucleotide to FBP to evaluate the cellular role of FBP. We have designed an antisense oligonucleotide of 24 residues that corresponds to a region around the translation initiation site, based the published complete nucleotide sequence and predicted amino acid sequence of a cDNA encoding human FBP. See E. Sadasivan et al., “Molecular cloning of the cDNA for a human folate binding protein,” Proc. Soc. Exp. Biol. Med., vol. 189, pp. 240-244 (1988). The targeted sense, and the designed antisense sequences are as follow:

| | | |
|---|---|---|
| Sense (coding)sequence: | 5′ GGG ACA GAC ATG GCT CAG CGG ATG 3′ | (SEQ ID NO:3) |
| Antisense sequence: | 5′ CAT CCG CTG TGC CAT GTC TGT CCC 3′ | (SEQ ID NO:4) |

The sense and antisense oligonucleotides are used in a time- and concentration-dependent experiment to test effects in the LNCAP, DU-145, and PC-3 prostate cancer cell lines, and in control non-cancerous prostate cancer cells.

### EXAMPLES 22-23

Antisense oligonucleotides to RCP. To the inventors' knowledge, there are no reports in the literature of using an antisense oligonucleotide to RCP to evaluate the cellular role of RCP. We have designed an antisense oligonucleotide of 24 residues that corresponds to a region around the translation initiation site, based the published complete nucleotide sequence and predicted amino acid sequence of a CDNA encoding human RCP. See D. Zheng et al., “Chicken riboflavin binding protein: cDNA sequence and homology with milk folate binding protein,” J. Biol. Chem., vol. 263, pp. 1126-1129 (1988). The targeted sense, and designed antisense sequences are as follow:

| | | |
|---|---|---|
| Sense sequence: | 5′-GGA ACA ACA ATG CTG AGG TTT GCC-3′ | (SEQ ID NO:5) |
| Antisense sequence: | 5′-GGC AAA CCT CAG CAT TGT TGT TCC-3′ | (SEQ ID NO:6) |

The sense and antisense oligonucleotides are used in a time- and concentration-dependent experiment to test effects in the LNCAP, DU-145, and PC-3 prostate cancer cell lines, and in control non-cancerous prostate cancer cells.

### EXAMPLES 24-35

Antisense experimental design and statistical significance. Each of the above antisense oligonucleotides for RBP, FBP, and RCP is tested in the LNCAP, DU-145, and PC-3 cell lines, as well as in a normal prostate cell line, at the following concentrations: 5, 10, 20, 40, and 80 μM. At each concentration, measurements are made after exposure times of 1, 2, 4, 8, 16, 24, 48, 72, and 96 hours. Non-treated and corresponding sense oligonucleotide-treated control groups are also measured for comparison purposes, to rule out possibilities such as non-specific, toxic effects attributable to the oligonucleotides. Each group is repeated in triplicate. Comparison with controls is evaluated using ANOVA and Student's ‘t’ test. Statistical significance is set at the P<0.05 level.

At the end of each measurement period, the cells are observed for changes in morphological characters such as rounding and attachment to substratum. Cell viability is tested by the trypan blue dye exclusion test under standard conditions. Numbers of viable and dead cells are counted using a hemocytometer. Cells are harvested and expression levels of the respective vitamin binding proteins are quantified using sensitive enzyme-linked immunoassays for each VBP. The results are expressed as concentration of VBP per 10⁶ cells. The culture medium is collected, cells are removed, and levels of PSA are quantified as a measure of the function of the cells, using a commercially available kit. Any apoptosis is assessed using the TUNEL and Cell Death ELISA plus kit, as described above, using the specific antibodies.

Expected results. We expect to observe dose- and time-dependent inhibition of cellular RBP, FBP, and RCP levels by their respective antisense oligodeoxynucleotides, but not by the sense oligonucleotides. Based on our preliminary results, following treatment with antisense oligonucleotide to RBP and FBP we expect to observe cell rounding, cell detachment, and loss of cell function as evidenced by reduction in or loss of PSA secretion. However the effects of inhibiting RCP expression may not be as pronounced as those for RBP and FBP, or they may appear at higher concentrations or after longer treatment times. We expect these experiments using antisense oligonucleotides to demonstrate the essential need for cellular over-expression of these VBPs to sustain cell division, function, and viability in prostate tumor cells.

These antisense oligonucleotides can be used to inhibit expression of VBPs in vivo, for example by systemic administration of the antisense oligonucleotides. Antisense oligonucleotides against other proteins have been effectively administered systemically. See, e.g., Dean et al., Brysch et al., and Colige et al. Antisense oligonucleotides may be made more stable, and more suitable for systemic administration, by using modified nucleotides known in the art, for example, phosphorothioate nucleotides. See, e.g., Dean et al.

### EXAMPLES 36-38

RNA Interference. As an alternative to the antisense approach described above, one could instead use the technique of RNA interference, in which double-stranded RNA oligonucleotides are administered to induce RNA interference (RNAi) or post-transcriptional gene silencing (PTGS). Small interfering double-stranded RNAs (siRNAs) will be produced, using portions of the coding sequences of RCP, RBP, or FBP. The siRNAs will inhibit expression of the VBPs in a manner analogous to that described above for antisense RNA sequences, though perhaps acting more efficiently.

RNAi techniques for silencing gene expression are known in the art. See, e.g., P. Sharp, “RNA Interference—2001,” Genes & Development, vol. 15, pp. 485-490 (2001); and Imgenex, “GeneSuppressor™ RNA Interference Kits,” available on the Internet on Mar. 11, 2003 at www.imgenex.com. Kits for implementing PTGS protocols are available commercially, for example, one of various GeneSuppressor™ kits sold by Imgenex Corp. (San Diego, Calif.).

### EXAMPLES 39-56

Effects of neutralizing RBP, FBP, or RCP on tumorigenesis and tumor progression in the nude mouse model. The effects of neutralizing each of the three VBPs separately or in combination are assessed on tumorigenicity and tumor progression in vivo in the athymic nude mouse model. About 5×10⁶ cells suspended in 100 microliters of Matrigel™ matrix (10 mg/mL) are injected subcutaneously into the flanks of each mouse. In prior experiments in our laboratory, such injections have produced tumors in over 90% of the animals. Tumor size is measured twice weekly. A minimum of ten mice are used per group. The animals are treated for up to eight weeks. At the end of treatment, the animals are sacrificed, and any remaining tumors are analyzed histologically. The tumor tissue sections are also evaluated for apoptosis by the TUNEL method, as previously described. The animals are weighed weekly during the experimental period. After sacrifice, the weights of spleen, liver, kidneys, and adrenal glands are recorded to assess any toxic effects.

Experimental design. The following treatments are performed with each of the three prostate cancer cell lines LNCAP, DU-145, and PC-3. These treatments enable the assessment of the effects of neutralizing the VBPs in both androgen-sensitive and androgen-insensitive cell lines.

| Group | Treatment |
|---|---|
| 1. Control | Non-immune gamma globulin |
| 2. Control | Anti-RBP gamma globulin after absorption with excess RBP |
| 3. Treated | Anti-RBP gamma globulin |
| 4. Control | Anti-FBP gamma globulin after absorption with excess FBP |
| 5. Treated | Anti-FBP gamma globulin |
| 6. Control | Anti-RCP after absorption with excess RCP |
| 7. Treated | Anti-RCP gamma globulin |
| 8. Control | Combination of absorbed antibodies to RBP, FBP, and RCP |
| 9. Treated | Combination of antibodies to RBP, FBP and RCP |

We administer excess levels of antibodies, levels that are capable of neutralizing all the circulating vitamin binding protein(s) under investigation in a particular group. Based on our preliminary results (where we found that we could prevent tumor formation by administering antibodies to VBPs), we estimate that these treatments may require twice-weekly injections of sufficient antibody to bind about 50 micrograms of each pertinent binding protein.

In one set of experiments, the antibody treatment begins at the same time as the injection of tumor cells, using the above 9 groups in the experimental design. These experiments assess the ability of these antibodies to prevent tumor formation, individually or in combination.

In another set of experiments, the effects of the antibodies on tumor growth and progression are evaluated by starting treatment when the tumors reach 0.5 mL in volume (about 2 weeks), again using the above 9 groups in the experimental design. These experiments assess the ability of the antibodies to inhibit tumor progression, or to shrink tumors.

Expected results. In the experiments where treatment begins at the same time as injection of the tumor cells, we expect to observe total inhibition of tumor formation by anti-RBP alone, by anti-FBP alone, or by a combination of antibodies to all 3 VBPs.

In preliminary results from our immunohistochemical experiments, RCP was observed to be moderately over-expressed in prostate adenocarcinoma. Further, RCP is an estrogen-induced protein. Thus it is possible that over-expression of RCP could depend on conversion of androgens to estrogens by aromatization in the prostate. Estrogens have been implicated in directing stromal proliferation and secretion, as well as in conditioning the response of the prostatic epithelium to androgens. We therefore expect to observe partial inhibition of tumor formation or growth following administration of the anti-RCP antibody.

In the experiments where treatment begins after the tumor has progressed to about 0.5 mL, we expect to observe a halt in the growth of the tumor, perhaps followed by regression, particularly in the groups 3, 5, and 9 above, those treated with anti-FBP and anti-RBP antibodies and with the combination. Synergies among the effects of the antibodies in a combination may result in greater or faster regression, as compared to treatment with an individual antibody alone. We also determine the extent to which apoptosis may be involved in antibody-induced tumor regression.

Data are analyzed statistically using ANOVA. Differences are considered significant at a P value of <0.05, in Student's ‘t’ test.

### EXAMPLES57-59

Vaccines. These VBPs or their constituent peptides may be used as vaccines for preventing or treating prostate cancer. Such vaccines, in addition to inducing the production of neutralizing antibodies, will also provoke a cellular immune response, T lymphocytes that specifically recognize and destroy cancer cells expressing vitamin binding proteins and their receptors. These cytotoxic T lymphocytes (CTLs) will specifically recognize cells over-expressing the VBPs and kill them. By analogy, it is known that CTLs against FBP may be induced. See, e.g., Kim et al. (1999) and Neglia et al. (1999).

As previously discussed, heterologous vitamin binding proteins may be used to stimulate an immune response. Potent antibody production has been demonstrated in the experimental data in rabbits reported above. Without wishing to be bound by this theory, it is believed that cell-mediated immunity is also stimulated by the administration of a heterologous vitamin binding protein as a vaccine. For example, heterologous RCP (chicken), FBP (bovine), and RBP (rodent) may be used as vaccines in humans. The resulting humoral and cell-mediated immune responses will attack cancers that depend on these VBPs, such as prostate cancer. The heterologous VBPs may be injected singly, or in combination, preferably adsorbed to a suitable adjuvant known in the art, for example, aluminum hydroxide gel or alum, to elicit a potent immune response that will also cross-react against the “self” VBPs.

When peptide vaccines are used, the peptides may correspond to sequences from VBPs from the same species, or to those from a different species, mammalian or non-mammalian. Whether the peptides are derived from the same species or a different species, as is known in the art a better immunological response will generally result when the peptide is conjugated to an immunogenic carrier protein, such as keyhole limpet hemocyanin, diphtheria toxoid, or cholera toxoid. Techniques for carrying out such conjugations, and for immunizations with protein conjugates generally are well known in the art. Alternatively, the peptide vaccines may be administered with anti-CTLA 4 to potentiate T cell response. CTLA 4 is a known negative co-stimulator of T cell response in inducing tumor immunity. See E. Davila et al., “Generation of Antitumor Immunity by Cytotoxic T Lymphocyte Epitope Peptide Vaccination, CpG-oligodeoxynucleotide Adjuvant, and CTLA-4 Blockade,” Cancer Res., vol. 15, pp. 3281-3288 (2003).

Examples of peptides from RCP that may be used in such vaccines include peptides taken from any part of the protein sequence, including amino acids 3-23, 64-83, 130-147, and 200-219 as shown in the sequence for chicken RCP found in FIG. 1 of S. Subramanian et al., “Expression, purification, and characterization of minimized chicken riboflavin carrier protein from a synthetic gene in Escherichia coli,” Protein Expression & Purification, vol. 26, pp. 284-289 (2002). These peptides were reported to elicit neutralizing antibodies capable of curtailing pregnancy in rodents. Additional examples of peptides from RCP that will be used, again with reference to the sequence as shown in FIG. 1 of S. Subramanian et al., are amino acids 33-49, and 169-186, which, even though they were not reported to induce pregnancy-neutralizing antibodies, are nevertheless immunodominant and are therefore candidates for inducing an immune response, such as a cellular immune response. Additional sequences from RCP that will be used include amino acids 23-33, 50-62, 84-94, 95-105, 106-120, 121-131, 148-158, 159-169, and 187-200. RCPs from other species might also be used.

The sequence for human RBP, as reported by the National Center for Biotechnology Information, Entrez Protein, www.ncbi.nlm.nih.gov, accession NP_{—}006735, is

| | | | | | | |
|---|---|---|---|---|---|---|
| 1 | ERDCRVSSFR | VKENFDKARF | SGTWYAMAKK | DPEGLFLQDN | IVAEFSVDET | (SEQ ID NO:7) |
| 51 | GQMSATAKGR | VRLLNNWDVC | ADMVGTFTDT | EDPAKFKMKY | WGVASFLQKG | |
| 101 | NDDHWIVDTD | YDTYAVQYSC | RLLNLDGTCA | DSYSFVFSRD | PNGLPPEAQK | |
| 151 | IVRQRQEELC | LARQYRLIVH | NGYCDGRSER | NL | | |

RBPs from other species might also be used. Examples of peptides from RBP that may be used in vaccines include peptides taken from any part of the protein sequence, including the peptides corresponding to amino acid residues 27-35, 60-70, and 89-101, as given in FIG. 1 of H. Melhus et al., “Epitope mapping of a monoclonal antibody that blocks the binding of retinol binding protein to its receptor,” Biochem. and Biophys. Res. Commun., vol. 210, pp. 105-112. Antibodies of these peptides have been reported to block RBP interaction with its receptor.

Peptides from FBP that may be used in vaccines include peptides taken from any part of the protein sequence, including those that elicit a strong cytotoxic T lymphocyte response. Different forms of FBP have been reported from different species, at least some of which also have a glycine methyl transferase activity, in addition to the folate binding activity. Any of these FBPs or their peptides may be used in practicing this invention; and, as previously stated, when peptides are used they may be from the same species or a different species from the mammal being immunized. For example, the sequence for Norwegian rat FBP, as reported by the National Center for Biotechnology Information, Entrez Protein, www.ncbi.nlm.nih.gov, accession 1XVA_A, is

| | | | | | | |
|---|---|---|---|---|---|---|
| 1 | VDSVYRTRSL | GVAAEGIPDQ | YADGEAARVW | QLYIGDTRSR | TAEYKAWLLG | (SEQ ID NO:8) |
| 51 | LLRQHGCHRV | LDVACGTGVD | SIMLVEEGFS | VTSVDASDKM | LKYALKERWN | |
| 101 | RRKEPAFDKW | VIEEANWLTL | DKDVPAGDGF | DAVICLGNSF | AHLPDSKGDQ | |
| 151 | SEHRLALKNI | ASMVRPGGLL | VIDHRNYDYI | LSTGCAPPGK | NIYYKSDLTK | |
| 201 | DITTSVLTVN | NKAHMVTLDY | TVQVPGAGRD | GAPGFSKFRL | SYYPHCLASF | |
| 251 | TELVQEAFGG | RCQHSVLGDF | KPYRPGQAYV | PCYFIHVLKK | TG | |

Another, the bovine milk folate binding protein, as reported by the National Center for Biotechnology Information, Entrez Protein, www.ncbi.nlm.nih.gov, accession P02702, is

| | | | | | | | |
|---|---|---|---|---|---|---|---|
| 1 | AQAPRTPRAR | TDLLNVCMDA | KHHKAEPGPE | DSLHEQCSPW | RKNACCSVNT | SIEAXKDISY | (SEQ ID NO:9) |
| 61 | LYRFNWDHCG | KMEPACKRHF | IQDTCLYECS | PNLGPWIREV | NQRWRKERVL | GVPLCKEDCQ | |
| 121 | SWWEDCRTSY | TCKSNWHKGW | NWTSGYNQCP | VKAAHCRFDF | YFPTPAALCN | EIWSHSYKVS | |
| 181 | NYSRGSGRCI | QMWFDPFQGN | PNEEVARFYA | ENPTSGSTPQ | GI | | |

Another, the human folate binding protein precursor, as reported by the National Center for Biotechnology Information, Entrez Protein, www.ncbi.nlm.nih.gov, accession A45753, is

| | | | | | | | |
|---|---|---|---|---|---|---|---|
| 1 | MAQRMTTQLL | LLLVWVAVVG | EAQTRIAWAR | TELLNVCMNA | KHHKEKPGPE | DKLHEQCRPW | (SEQ ID NO:10) |
| 61 | RKNACCSTNT | SQEAHKDVSY | LYRFNWNHCG | EMAPACKRHF | IQDTCLYECS | PNLGPWIQQV | |
| 121 | DQSWRKERVL | NVPLCKEDCE | QWWEDCRTSY | TCKSNWHKGW | NWTSGFNKCA | VGAACQPFHF | |
| 181 | YFPTPTVLCN | EIWTHSYKVS | NYSRGSGRCI | QMWFDPAQGN | PNEEVARFYA | AAMSGAGPWA | |
| 241 | AWPFLLSLAL | MLLWLLS | | | | | |

Another, a tentative sequence for bovine folate binding protein, as reported by the National Center for Biotechnology Information, Entrez Protein, www.ncbi.nlm.nih.gov, accession BFBO, is

| | | | | | | | |
|---|---|---|---|---|---|---|---|
| 1 | AQAPRTPRAR | TDLLNVCMDA | KHHKAEPGPE | DSLHEQCSPW | RKNACCSVNT | SIEAXKDISY | (SEQ ID NO:11) |
| 61 | LYRFNWDHCG | KMEPACKRHF | IQDTCLYECS | PNLGPWIREV | NQRWRKERVL | GVPLCKEDCQ | |
| 121 | SWWEDCRTSY | TCKSNWHKGW | NWTSGYNQCP | VKAAHCRFDF | YFPTPAALCN | EIWSHSYKVS | |
| 181 | NYSRGSGRCI | QMWFDPFQGN | PNEEVARFYA | ENPTSGSTPQ | GI | | |

Another, human folate binding protein, as reported by the National Center for Biotechnology Information, Entrez Protein, www.ncbi.nlm.nih.gov, accession AAB81937, is

| | | | | | | | |
|---|---|---|---|---|---|---|---|
| 1 | MLPAATEVQH | RLQGQKDMVW | KWMPLLLLLV | CVATMCSAQD | RTDLLNVCMD | AKHHKTKPGP | (SEQ ID NO:12) |
| 61 | EDKLHDQCSP | WKKNACCTAS | TSQELHKDTS | RLYNFNWDHC | GKMEPACSAT | SSRTPVSMSA | |
| 121 | HQPGALDPAG | ESELAAKNAS | WMCPYAKSTV | SAGGRIVTPP | TRARATGTED | GTGPQELTSA | |
| 181 | QLGLSAAPLS | PTSPLQLPFV | KASGVTHTRS | ATTAEGAAAA | SRCGLLQPRA | TPTRKWRGSM | |
| 241 | LQPCM | | | | | | |

A predicted amino acid sequence for folate binding protein from the SKOV3 human ovarian cancer cell line may be found in FIG. 1 of I. Campbell et al., “Folate binding protein is a marker for ovarian cancer,” Cancer Res., vol. 51, pp. 5329-5338 (1991).

### EXAMPLE 60

A similar “indirect” method may also be used against prostate cancers. In the “indirect” method, an unrelated, but immunogenic molecule (e.g., fluorescein isothiocyanate) is used to induce an immune response in the host. Thereafter, a conjugate of folate, retinol, or riboflavin with the same immunogenic molecule is administered to the host. The retinol or riboflavin component of the conjugate binds to the corresponding VBP, which is disproportionately found on membranes of cancer cells. The antibodies against the immunogen then target the cells to which the conjugate has bound, attracting immune system cells such as T-cells to home in on and destroy the tumor cells. This method is analogous in some respects to that disclosed in published U.S. patent application Ser. No. 2001/0031252. Other immunogenic compounds known in the art may be used in lieu of fluorescein isothiocyanate. Advantages of fluorescein isothiocyanate are that it is unrelated to anything that is naturally present in mammals; that it is immunogenic; and that it is easy to track due to its fluorescent properties.

Miscellaneous

Humanized antibodies developed by recombinant techniques, either individually or in combination; or bispecific antibodies coupled to various agents; or both may be used in immunotherapy of hormone-sensitive and -resistant prostate cancers.

It is possible that there could be some long-term toxicity associated with the practice of this invention, but it is believed that the beneficial anti-cancer effects should outweigh the toxic effects. Such considerations are not uncommon in cancer therapies, and it is expected that any toxicity that might be associated with the present invention should be less than that found with many existing forms of chemotherapy. For example, we have noted that rabbits that have been immunized against RCP, RBP, or FBP for over two years have died of natural causes, and did not show any toxic symptoms such as weight loss or increased mortality. However, since RBP is involved in carrying vitamin A to tissues in the eye, and since vitamin A is required for normal vision, it is possible that passive or active immunization against RBP could be implicated in the induction of uveitis or other pathologies of the eye. In addition, immunization against any of these three VBPs may show reproductive toxicity by inducing sterility or abortion. RCP immunization has been studied to explore immunological methods of fertility regulation, and it has been observed that RCP antibodies administered to rats and monkeys will terminate pregnancy. These antibodies have also been shown to induce loss of spermatogenesis in male monkeys, but without apparent general toxicity. Furthermore, even to the extent that some toxicity may associated with the practice of this invention, other therapeutic compounds currently used to treat cancers have substantially greater toxicity—e.g., hair loss, fatigue, nausea, weight loss, etc. These side effects are generally accepted as “the lesser of two evils.” Having said this, it is not currently known what long term effects might result in humans from the long-term use of this invention, and any toxic effects will need to be assessed and evaluated in considering the therapeutic use of this invention in cancer patients.

Compounds in accordance with the present invention may be administered to a patient for treatment of prostate cancer by any suitable means, including oral, intravenous, parenteral, subcutaneous, intrapulmonary, and intranasal administration. Parenteral infusions include intramuscular, intravenous, intraarterial, or intraperitoneal administration. The compounds may also be administered transdermally, for example in the form of a slow-release subcutaneous implant, or orally in the form of capsules, powders, or granules. They may also be administered by inhalation.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers dextrose, dextrose and sodium chloride, lactated Ringers, or fixed oils. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampule, each containing a unit dose amount, or in the form of a container containing multiple doses.

The compounds may be formulated into therapeutic compositions as pharmaceutically acceptable salts. These salts include acid addition salts formed with inorganic acids, for example hydrochloric or phosphoric acid, or organic acids such as acetic, oxalic, or tartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

Controlled delivery may be achieved by admixing the active ingredient with appropriate macromolecules, for example, polyesters, polyamino acids, polyvinyl pyrrolidone, ethylenevinylacetate, methylcellulose, carboxymethylcellulose, prolamine sulfate, or lactide/glycolide copolymers. The rate of release of the active compound may be controlled by altering the concentration of the macromolecule.

Another method for controlling the duration of action comprises incorporating the active compound into particles of a polymeric substance such as a polyester, peptide, hydrogel, polylactide/glycolide copolymer, or ethylenevinylacetate copolymers. Alternatively, an active compound may be encapsulated in microcapsules prepared, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

Initial in vivo animal trials will be conducted in accordance with all applicable laws and regulations, following by clinical trials in humans in accordance with all applicable laws and regulations.

As used in the specification and claims, an “effective amount” of an antagonist to a vitamin binding protein is an amount sufficient to prevent tumor or metastasis formation, or to inhibit the formation of tumors or metastases, or to reduce the number or size of tumors or metastases, to a clinically significant degree. An “effective amount” of a vaccine comprising a heterologous vitamin binding protein is an amount of the heterologous vitamin binding protein that, when injected into a patient (with or without, preferably with, a suitable adjuvant) is sufficient to prevent tumor or metastasis formation, or to inhibit the formation of tumors or metastases, or to reduce the number or size of tumors or metastases, to a clinically significant degree. “Significance” for these purposes is determined as the P<0.05 level, or by such other measure of statistical significance as is commonly used in the art for a particular type of determination.

The complete disclosures of all references cited in this specification are hereby incorporated by reference. In the event of an otherwise irreconcilable conflict, however, the present specification shall control.

What is claimed:

1. A method for inhibiting prostate cancer in a mammal, said method comprising administering to the mammal an effective amount of one or more antagonists to one or more compounds selected from the group consisting of riboflavin carrier protein, folic acid binding protein, and retinol binding protein.

2. A method as recited in claim 1, wherein the one or more antagonists comprise one or more antibodies that selectively bind to one or more compounds as recited.

3. A method as recited in claim 1, wherein the one or more antagonists comprise one or more antisense oligonucleotides that inhibit translation of one or more compounds as recited.

4. A method as recited in claim 3, wherein said antisense oligonucleotides comprise modified nucleotides that render the oligonucleotides more resistant to in vivo degradation by RNase than are otherwise identical oligonucleotides consisting only of unmodified nucleotides.

5. A method as recited in claim 1, wherein the one or more antagonists comprise one or more double-stranded RNA oligonucleotides that inhibit translation of one or more compounds as recited through RNA interference or post-translational gene silencing.

6. A method as recited in claim 1, wherein the one or more antagonists comprise an antagonist to riboflavin carrier protein.

7. A method as recited in claim 1, wherein the one or more antagonists comprise an antagonist to folic acid binding protein.

8. A method as recited in claim 1, wherein the one or more antagonists comprise an antagonist to retinol binding protein.

9. A method for inhibiting prostate cancer in a mammal afflicted with prostate cancer, said method comprising immunizing the mammal against one or more compounds selected from the group consisting of heterologous riboflavin carrier protein, heterologous folic acid binding protein, and heterologous retinol binding protein.

10. A method as recited in claim 9, wherein said method comprises immunizing the mammal against heterologous riboflavin carrier protein.

11. A method as recited in claim 9, wherein said method comprises immunizing the mammal against heterologous folic acid binding protein.

12. A method as recited in claim 9, wherein said method comprises immunizing the mammal against heterologous retinol binding protein.

13. A method for preventing prostate cancer in a mammal that is not known to be afflicted with prostate cancer, said method comprising immunizing the mammal against one or more compounds selected from the group consisting of heterologous riboflavin carrier protein, heterologous folic acid binding protein, and heterologous retinol binding protein.

14. A method as recited in claim 13, wherein said method comprises immunizing the mammal against heterologous riboflavin carrier protein.

15. A method as recited in claim 13, wherein said method comprises immunizing the mammal against heterologous folic acid binding protein.

16. A method as recited in claim 13, wherein said method comprises immunizing the mammal against heterologous retinol binding protein.

17. A method for inhibiting prostate cancer in a mammal, said method comprising administering to the mammal an immunogen in an amount and for a time sufficient to stimulate production of antibodies against the immunogen; and subsequently administering to the mammal an effective amount of a conjugate of the immunogen and a vitamin selected from the group consisting of riboflavin, folate, and retinol; wherein the vitamin component of the conjugate causes the conjugate to bind preferentially to the membranes of prostate cancer cells; and wherein the immunogen component of the conjugate causes the mammal's immune system to attack prostate cancer cells to which the vitamin component has bound.

18. A method as recited in claim 17, wherein the vitamin is riboflavin.

19. A method as recited in claim 17, wherein the vitamin is folate.

20. A method as recited in claim 17, wherein the vitamin is retinol.

21. A method as recited in claim 17, wherein the immunogen is fluorescein isothiocyanate.

22. A method for inhibiting prostate cancer in a mammal afflicted with prostate cancer, said method comprising immunizing the mammal against one or more compounds selected from the group consisting of peptide subunits of riboflavin carrier protein, peptide subunits of folic acid binding protein, and peptide subunits of retinol binding protein; wherein the peptide subunits may correspond to subunits of proteins from the same species or from a different species as that to which the mammal belongs.

23. A method for preventing prostate cancer in a mammal that is not known to be afflicted with prostate cancer, said method comprising immunizing the mammal against one or more compounds selected from the group consisting of peptide subunits of riboflavin carrier protein, peptide subunits of folic acid binding protein, and peptide subunits of retinol binding protein; wherein the peptide subunits may correspond to subunits of proteins from the same species or from a different species as that to which the mammal belongs.
